(19) United States
(12) Patent Application Publication
(10) Pub. No.: US 2023/0173145 A1
(43) Pub. Date: Jun. 8, 2023

(54) PEPTOID-CONTAINING PERSONAL LUBRICANT

(71) Applicant: MAXWELL BIOSCIENCES, INC., Austin, TX (US)

(72) Inventors: ANNELISE E. BARRON, Woodside, CA (US); JOSHUA MCCLURE, Austin, TX (US)

(21) Appl. No.: 17/906,811

(22) PCT Filed: Dec. 18, 2020
(86) PCT No.: PCT/US2020/065777

§ 371 Date: Sep. 20, 2022

Related U.S. Application Data
(60) Provisional application No. 62/948,967, filed on Dec. 17, 2019.

(51) Int. Cl. A61L 31/16; A61L 31/10; A61K 38/08; A61P 31/12

(52) U.S. Cl. CPC A61L 31/16; A61L 31/10; A61K 38/08; A61P 31/12; A61L 2400/10; A61L 2300/408; A61L 2300/25

(57) ABSTRACT

A contraceptive device is provided which includes a (preferably elastomeric) surface; and a lubricant applied to the surface. The lubricant includes (a) a lubricious medium, and (b) a pharmaceutically effective amount of a peptoid disposed in said lubricious medium. The antimicrobial peptoid may impart protection against some common sexually transmitted diseases, while also imparting spermicidal activity to the lubricious medium and contraceptive device.

### CROSS-REFERENCE TO RELATED APPLICATIONS

This application claims the benefit of U.S. provisional application No. 62/948,967, filed Dec. 17, 2019, having the same title, and having the same inventors, and which is incorporated herein by reference in its entirety.

### FIELD OF THE DISCLOSURE

The present disclosure relates generally to antimicrobial compositions, and more particularly to a personal lubricant, and to a condom or other contraceptive device treated with the same, which contains an antimicrobial peptoid composition.

### BACKGROUND OF THE DISCLOSURE

Within the last couple of decades, a significant amount of research has focused on the use of antimicrobial peptides in the treatment of multi-drug resistant bacteria. Natural antimicrobial peptides (AMPs) are known to defend a wide array of organisms against bacterial invaders. These AMPs have shown potential as supplements for (or replacements of) conventional antibiotics, since few bacteria have evolved resistance to them.

AMPs destroy bacteria in various ways. Some AMPs kill bacteria by permeating the cytoplasmic membrane and causing depolarization or leakage of internal cell materials. Other AMPs function by targeting anionic bacterial constituents, such as DNA, RNA, or cell wall components. Bacterial resistance to AMPs is rare, possibly because AMPs have evolved along with the resistance mechanisms that are designed to evade them. Moreover, the targets of many AMPs (such as bacterial plasma membranes and anionic intracellular macromolecules) are sufficiently general that changes to the sequence of the AMP can be made to subvert resistance, without having any significant adverse impact on overall functionality.

Although AMPs have been actively studied for decades, they have yet to achieve widespread clinical use. This is due, in part, to the vulnerability of many peptide therapeutics to rapid in vivo degradation, which dramatically reduces their bioavailability.

The foregoing problems have led to the development of peptidomimetics, which are small, protein-like chains designed to mimic a peptide. Peptidomimetics may be made by modifying an existing peptide, or may be based on similar systems that mimic peptides, such as peptoids and β-peptides.

Peptoids (poly-N-substituted glycines) are isomers of peptides in which side chains are attached to the backbone amide nitrogen rather than to the α-carbon. Antimicrobial peptoids have been described, for example, in U.S. Pat. No. 8,445,632 (Barron et al.), entitled “Selective Poly-N-Substituted Glycine Antibiotics”, which is incorporated herein by reference in its entirety.

Peptoids are particularly well-suited for AMP mimicry. Peptoids are easily synthesized using conventional peptide synthesis equipment, and provide access to diverse sequences at relatively low cost. Submonomer synthetic methods are known that may be utilized to impart a wide variety of chemical functionalities to peptoids. Consequently, peptoids are highly and finely tunable. Furthermore, they are protease-resistant, and can be designed to form amphipathic helices that resist thermal and chaotropic denaturation.

### SUMMARY OF THE DISCLOSURE

In one aspect, a contraceptive device is provided which comprises a (preferably elastomeric) surface; and a lubricant applied to said surface, said lubricant including (a) a lubricious medium, and (b) a pharmaceutically effective amount of a peptoid disposed in said lubricious medium.

In another aspect, a method is provided for preventing the transmission of sexually transmitted diseases during sexual intercourse between two sexual partners. The method comprises providing a lubricant including (a) a lubricious medium, and (b) a pharmaceutically effective amount of a peptoid disposed in said lubricious medium; and applying the lubricant to (a) a contraceptive device, or (b) the genitalia of at least one of the partners.

### BRIEF DESCRIPTION OF THE DRAWINGS

FIG. 1 is a chart illustrating the efficacy of various peptoids against HSV-1 virus, as compared to LL-37 and a control.

FIG. 2 depicts the structure of Peptoid 1 [H—(NLys-Nspe-Nspe)₄-NH2, where NLys is N-(4-aminobutyl)glycine and Nspe is N—(S)-(1-phenylethyl)glycine].

FIGS. 3-8 depict the structures of several particular, non-limiting examples of halogenated analogs of Peptoid 1.

FIG. 9 depicts the structure of a shorter, modified version of Peptoid 1.

FIGS. 10-12 depict the structures of some particular, non-limiting examples of halogenated analogs of the peptoid whose structure is depicted in FIG. 8.

### DETAILED DESCRIPTION

Sexually transmitted diseases are a continuing concern for the human population. Many sexually transmitted diseases have become widespread, and some of these diseases currently lack an effective cure or treatment. Even among those sexually transmitted diseases for which cures or treatments have been developed, the emerging drug resistance of the underlying pathogens has become problematic.

The history of the herpes simplex virus (HSV) provides a sobering illustration of the foregoing issues. This virus, which is a member of the Herpesviridae family, is a significant human pathogen that results in mucocutaneous lesions in the oral cavity or genital infections. An estimated 60%-95% of the population is infected by at least one of these viruses, thus making HSV infections among the most common of human diseases. HSV infections are characterized by mild to severe symptoms, such as cold sores, keratitis, corneal blindness, and encephalitis. HSV infections increase the risk to patients of developing human immunodeficiency virus (HIV) infections, and thus contribute to the HIV epidemic.

At present, no current treatments exist which can eradicate an HSV infection, and the treatment of an oral or genital HSV infection does not prevent subsequent chronic infection of nerves. During recurrences, antiviral drugs, such as acyclovir, valacyclovir, or famciclovir, may provide limited relief and may resolve symptoms slightly faster (e.g., a day or two sooner) compared to untreated cases. however, even then, the efficacy of these treatments may depend on their prompt administration (e.g., within a few hours after the onset of symptoms). For patients suffering from frequent, painful attacks associated with HSV, suppressive therapy (the administration of antiviral drugs every day on an indefinite basis) can reduce the number of outbreaks. Notably, however, the administration of antiviral drugs (as part of suppressive therapy or otherwise) does not prevent infected individuals from transmitting the infection to others.

There is thus a need in the art for a means for preventing the spread of sexually transmitted diseases at the point of contact. There is also a need in the art for such a means which is effective against HSV and other viral pathogens. There is further a need in the art for such a means which selectively exposes pathogens of the type indicated in many sexually transmitted diseases to effective amounts of a composition which will destroy or deactivate the pathogen without also imparting drug resistance to it. These and other needs may be met with the compositions and methodologies disclosed herein.

It has now been found that some or all of the foregoing needs may be met through the provision of a personal lubricant comprising peptoid (oligomers of N-substituted glycines) compositions of the type disclosed herein. In some embodiments, this personal lubricant may be used in conjunction with, and is more preferably applied to at least one surface of, a contraceptive device. Possible contraceptive devices may include condoms (male or female), sponges, rings, diaphragms, implants or intrauterine devices. In an especially preferred embodiment, the contraceptive device is a condom, and the personal lubricant is applied to the sheath of the condom. However, other uses of this type of personal lubricant with other devices (such as, for example, adult novelty items) or as a standalone lubricant are also contemplated.

Peptoid compositions of the type disclosed herein have been found to exhibit surprising and unexpected activity against various microbial pathogens, including viruses. Without wishing to be bound by theory, the surprising effectiveness of at least some peptoids against viral pathogens is believed to arise from an apparent equivalence of mechanism of their antiviral activity to the Human Cathelicidin antimicrobial peptide LL-37. In particular, like LL-37, these peptoids exhibit a similar ability to pass through viral membranes and to bind to DNA or RNA. Consequently, these peptoids offer potential efficacy against the same viruses that LL-37 is active against including, without limitation, HSV-1, HSV-2, Vaccinia virus, Respiratory Syncytial Virus (RSV), the Hepatitis C Virus (HCV), influenza A viruses (IAV), and the HIV-1 virus. Peptoid compositions of the type disclosed herein are inherently less susceptible to the development of resistance by viral pathogens, have low toxicity, and do not undergo rapid in vivo degradation.

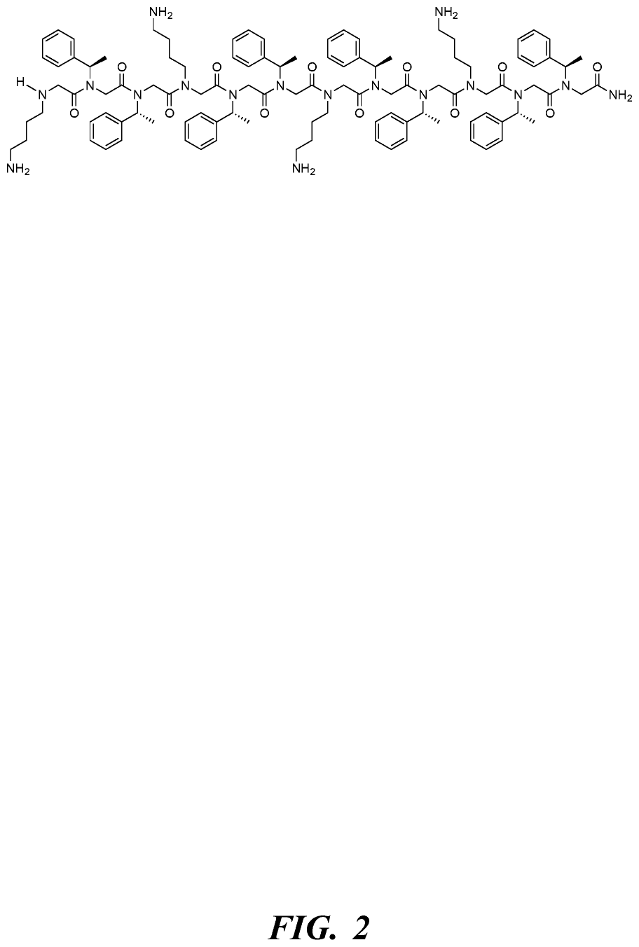

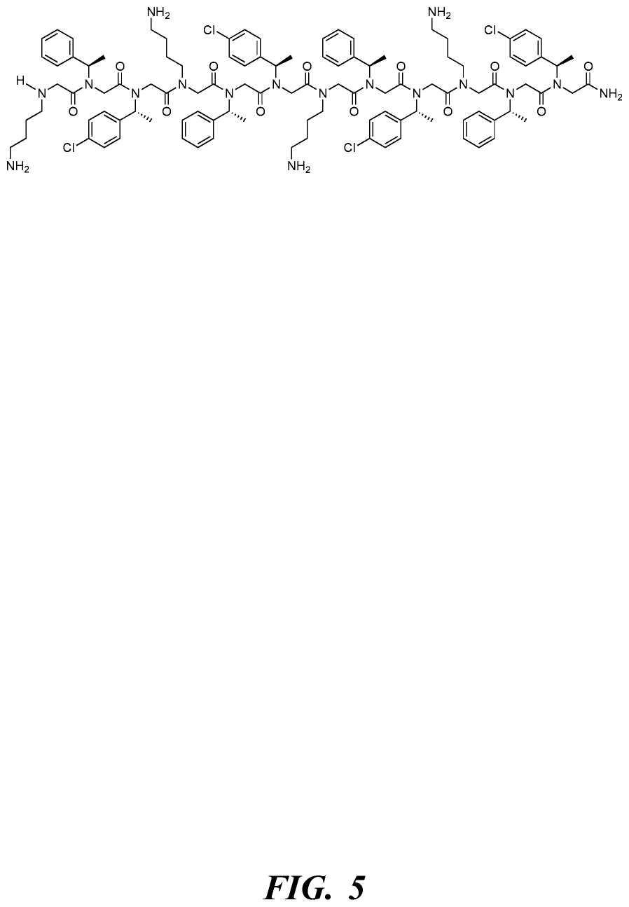

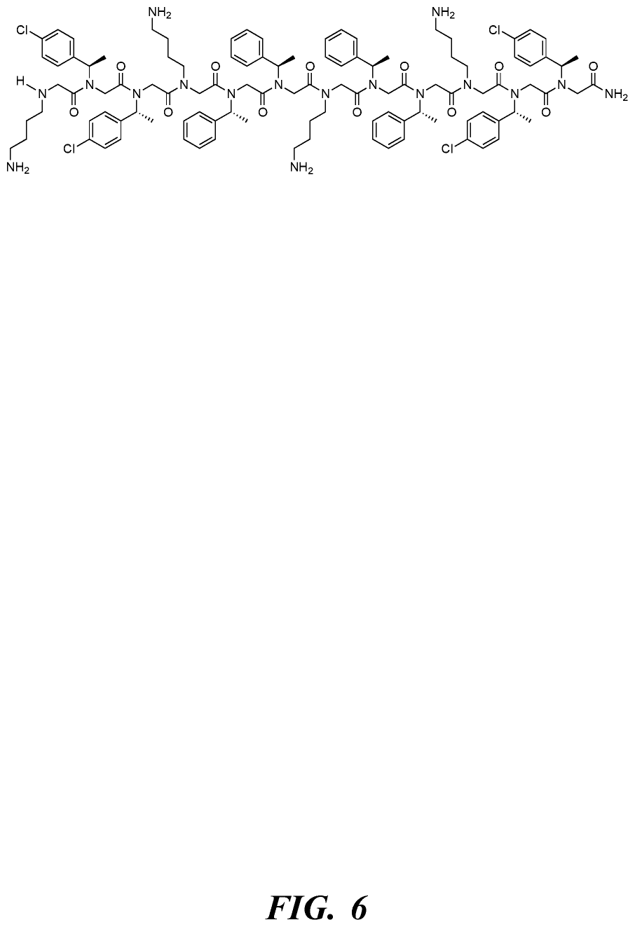

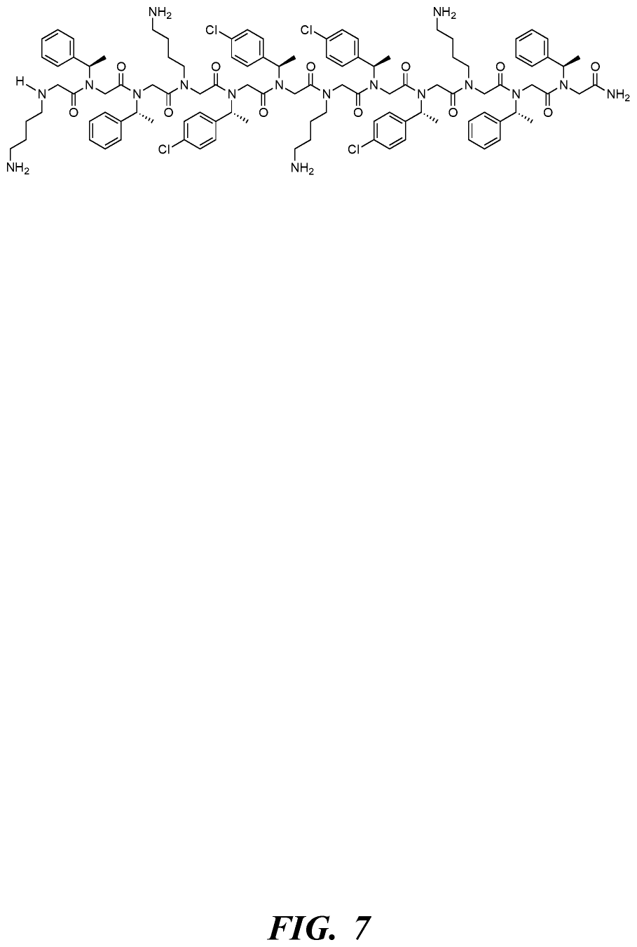

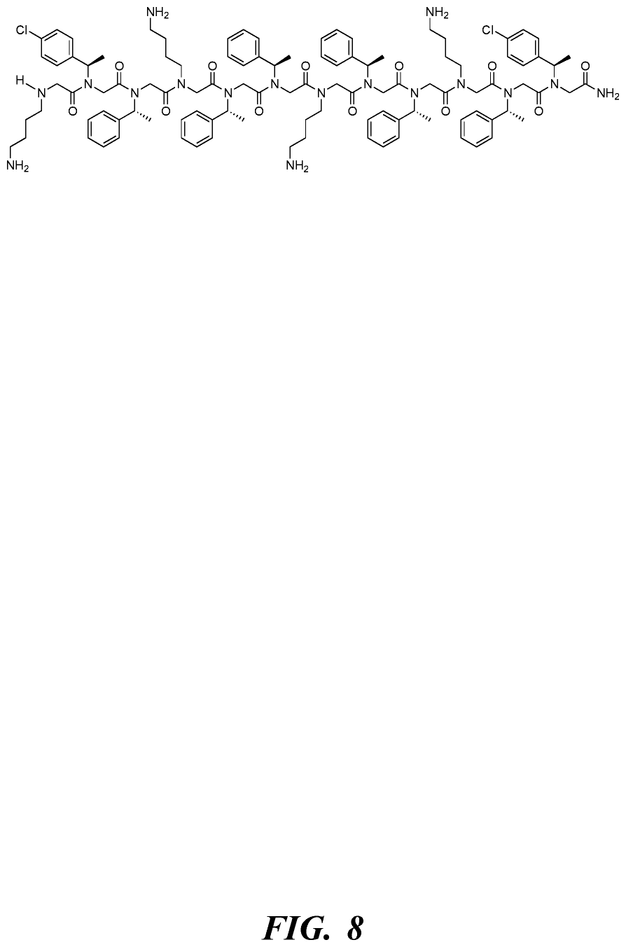

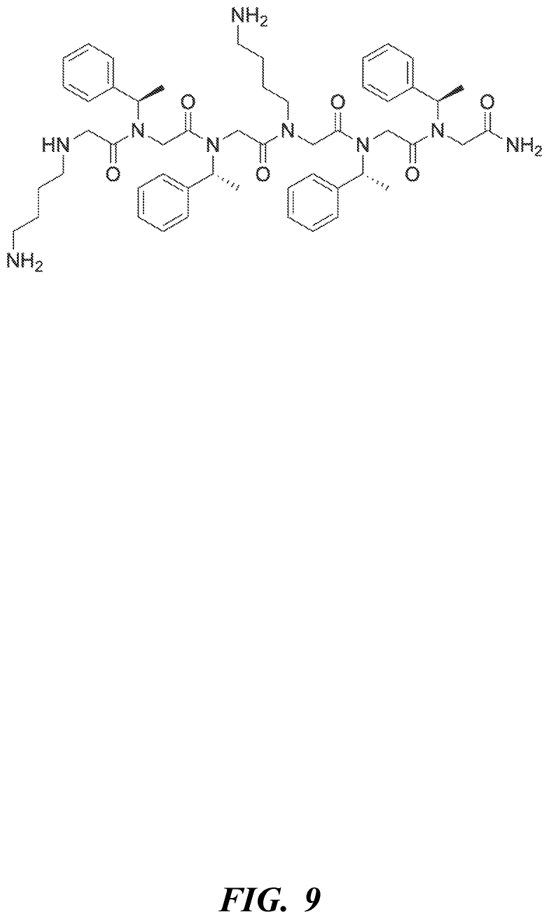

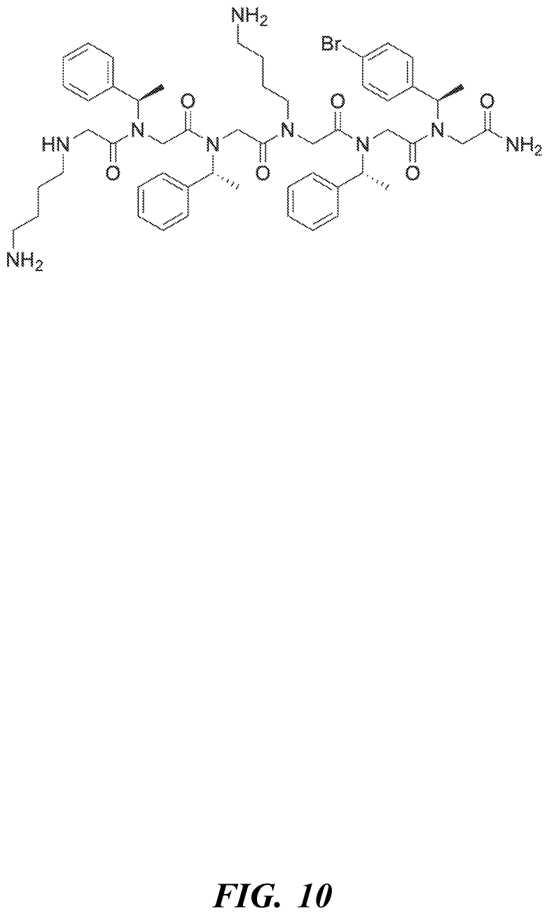

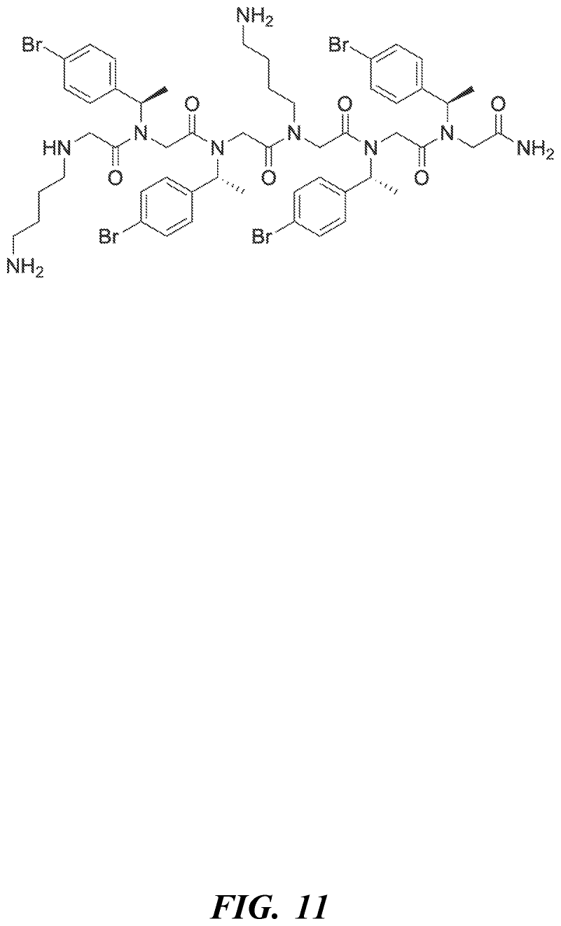

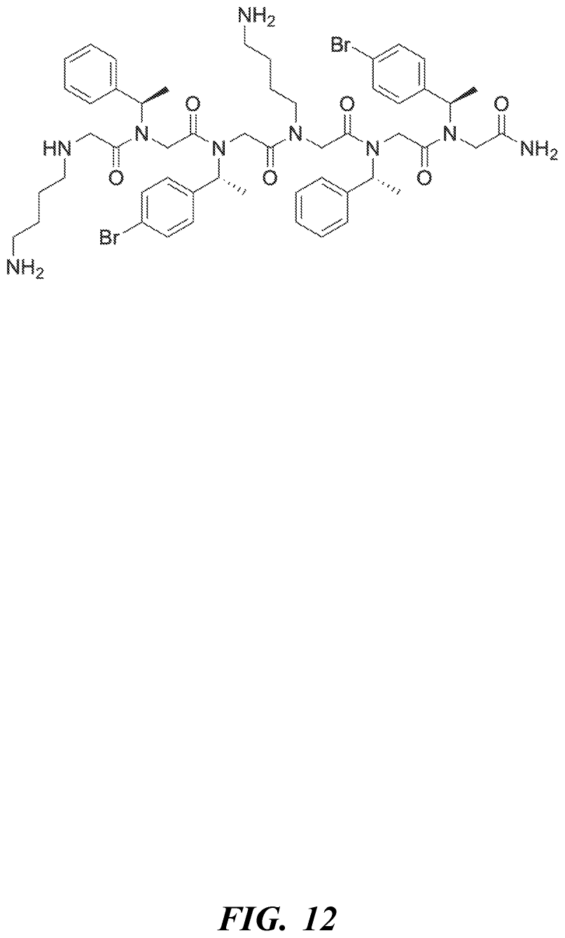

TABLE 1 lists some preferred embodiments of peptoids which may be utilized in the personal lubricants disclosed herein. The structures of some of these peptoids are depicted in FIGS. 2-12.

TABLE 1
Peptoid Samples

| compound | Reference | project |
|---|---|---|
| H—(NLys-Nspe-Nspe)₄-NH₂ | TM1 | Peptoid 1 |
| Cy5.5-Ahx-(NLys-Nspe-Nspe)₄-NH₂ | TM1-Cy5.5 | |
| H—(NLys-Nspe-Nspe(p-Br))₂—NH₂ | TM2 | |
| H-NLys-Nspe-Nspe-NLys-Nspe-Nspe(p-Br)—NH₂ | TM3 | |
| H—(NLys-Nspe(p-Br)-Nspe(p-Br))₂—NH₂ | TM4 | |
| H-Ntridec-NLys-Nspe-Nspe-NLys-NH₂ | TM5 | |
| H—(NLys-Nspe-Nspe)₃-NLys-Nspe-NH₂ | TM6 | |
| H—(NLys-Nspe-Nspe)₂-NH₂ | TM7 | |
| H-Ndec-(NLys-Nspe-Nspe)₂-NH₂ | TM8 | |
| H-Ndec-(NLys-Nspe-Nspe(p-Br))₂—NH₂ | TM9 | |
| H-Ntridec-(NLys-Nspe-Nspe(p-Br))₂—NH₂ | TM10 | |
| H—(NLys-Nspe-Nspe)₄-NLys-NH₂ | TM11 | Peptoid1 + NLys |
| H—(NLys-Nspe-Nspe)₃-NLys-Nspe-NLys-NH₂ | TM16 | |
| H—(NLys-Nspe-Nspe)₂-NLys-NH₂ | TM17 | |
| H-NLys-Nspe-Nspe-NLys-Nspe-Nspe(p-Br)-NLys-NH₂ | TM13 | |
| H—(NLys-Nspe-Nspe(p-Br))₂-NLys-NH₂ | TM12 | |
| H—(NLys-Nspe(p-Br)-Nspe(p-Br))₂-NLys-NH₂ | TM14 | |
| H-Ntridec-NLys-Nspe-Nspe-NLys-NLys-NH₂ | TM15 | |
| H-Ndec-(NLys-Nspe-Nspe)₂-NLys-NH₂ | TM18 | |
| H-Ndec-(NLys-Nspe-Nspe(p-Br))₂-NLys-NH₂ | TM19 | |
| H-Ntridec-(NLys-Nspe-Nspe(p-Br))₂-NLys-NH₂ | TM20 | |
| H—(NLys-NLeu-NLeu)₃-NLys-NLeu-NH₂ | TM21 | controls |
| H—(NLys-Nssb-Nssb)₄-NH₂ | TM22 | |

Various peptoids and oligomers of N-substituted glycines may be utilized in accordance with the teachings herein to make peptoid-containing personal lubricants. In addition to the peptoids set forth in TABLE 1, these include the peptoids described in U.S. Pat. No. 8,445,632 (Barron et al.), which is incorporated herein by reference in its entirety, as well as the peptoids disclosed in U.S. Pat. No. 9,938,321 (Kirshenbaum et al.), U.S. Pat. No. 9,315,548 (Kirshenbaum et al.) and U.S. Pat. No. 8,828,413 (Kirshenbaum et al.), all of which are incorporated herein by reference in their entirety.

Various halogenated peptoids and halogenated oligomers of N-substituted glycines may also be utilized in accordance with the teachings herein to make peptoid-containing personal lubricants. These include, without limitation, various halogenated analogs of the foregoing peptoids and oligomers of N-substituted glycines, including those disclosed in WO2020223581 (Molchanova et al.), which is incorporated herein by reference in its entirety. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. However, the use of one or two bromo- or chloro-substitutions is preferred in many applications. Moreover, while the peptoids described herein may be halogenated at various locations, para halogenation on the aryl rings of peptoids including such moieties is especially preferred in many applications, although ortho- and meta-substitution, or even perhalogenation, may be useful in some applications.

The peptoid compositions described herein may also be alkylated, and preferably have terminal alkylation. Here, alkylation (and especially terminal alkylation) with a C10 or C13 tail is especially preferred. It has been found that such terminal alkylation can dramatically enhance the antibacterial activity of a peptoid, and in some cases, may cause a peptoid which otherwise has low antibacterial activity to have significant antibacterial activity.

The personal lubricants disclosed herein may comprise various ingredients. These may include, without limitation, purified water, glycerin, propylene glycol, polyquaternium 15, methylparaben, propylparaben, hydroxyethylcellulose, caprylyl glycol, caprylhydroxamic acid, propanediol, lactic acid, dimethicone, cyclomethicone, dimethicone/vinyl dimethicone crosspolymer, caprylic/capric triglyceride, xylitol, aloe barbadensis leaf juice, pectin, Chamomilla recutita (matricaria) flower extract, potassium ascorbyl tocopheryl phosphate (Vitamins C & E), phenoxyethanol, sodium gluconate, sodium saccharin, sodium benzoate, citric acid, fructose, galactose, potassium phosphate, sodium phosphate, sodium hydroxide, propanediol, gluconolactone, phenoxyethanol, cellulose gum, cyclopentasiloxane, sodium polyacrylate trideceth-6, PEG/PPG-18/18 dimethicone, organic aloe barbadensis leaf juice, xanthan gum, natural flavors, potassium sorbate, carrageenan, dimethyl isosorbide, organic Cannabis sativa seed oil, cellulose gum, EDTA, carbomer, PEG-90M, tetrahydroxypropyl ethylenediamine, potassium sorbate, cyclopentasiloxane, sorbitan stearate, isopropyl myristate, cetearyl alcohol, polysorbate 60, hydrolyzed silk, sodium hyaluronate, cyclopentasiloxane, PEG-45M, sucralose, sodium chloride, citric acid, cyclopentasiloxane, cyclotetrasiloxane, dimethiconol, sorbitol, polysorbate 60, hydroxyethylcellulose, benzoic acid, tocopheryl acetate, maltodextrin, sucralose, chlorhexidine gluconate, gluconolactone, sodium hydroxide, maltodextrin, sucralose, menthyl lactate, methyl salicylate, Glycerin, Propylene Glycol, Maltodextrin, Honey, Methylparaben, Sucralose, polycarbophil, carbomer homopolymer (Type B), ethylparaben sodium, methylparaben sodium, propylparaben sodium, hypromellose, sodium chloride, sodium phosphate, disodium phosphate, potassium chloride, magnesium chloride, calcium chloride, Prunus dulcis (sweet almond) oil, butyrospermum parkii (Shea butter), Helianthus annuus (sunflower) seed oil, Theobroma cacao (cocoa) seed butter, cera alba (bees wax), and natural tocopherols (vitamin E).

The use of anionic microgels in the personal lubricants described herein is especially preferred. In such embodiments, one or more peptoids may be loaded into the microgel as, for example, by complexation. The peptoids used for this purpose may be selected based on their spermicidal and/or antiviral properties.

In some embodiments, the personal lubricants disclosed herein may be used in combination with a condom which includes a polyacrylic-acid coated surface. In such embodiments, one or more peptoids may be released from the polyacrylic-acid coated surface.

Various counterions may be utilized in forming pharmaceutically acceptable salts of the materials disclosed herein. One skilled in the art will appreciate that the specific choice of counterion may be dictated by various considerations. However, the use of sodium and hydrochloride salts may be preferred in some applications.

In some embodiments, the compositions described herein may be formulated as mixtures of two or more peptoids. These mixtures may feature peptoids in various ratios. For example, in some embodiments, a first peptoid with higher antiviral efficacy but higher cytotoxicity may be mixed with a second peptoid of lower antiviral efficacy and lower cytotoxicity to produce a mixture with acceptable levels of efficacy and cytotoxicity.

### Example 1

This example illustrates the surprising efficacy of several peptoids against the HSV-1 virus.

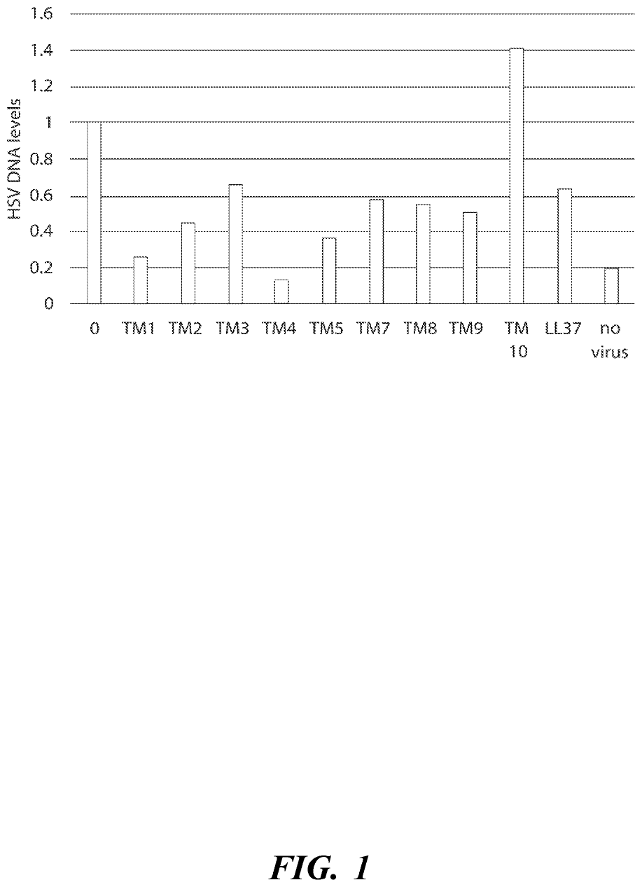

A series of 9 peptoids were tested for activity against HSV-1. The peptoids were incubated with 10⁵ pfu HSV-1 GFP for 2 hours at 37° C. Virus was added to triplicate cultures of OKF6/TERT-1 cells (oral keratinocytes) at an MOI of 0.01:1, and incubated for a further 24 hours at 37° C. Total DNA was isolated from the cultures, and relative HSV-1 DNA levels were quantified by QPCR relative to genomic β-actin. The results are shown in FIG. 1 (see TABLE 1 for peptoid information). As seen therein, there was varied activity among the peptoids against HSV-1.

### Example 2

This example illustrates the time and dose-dependence of peptoids against HSV-1.

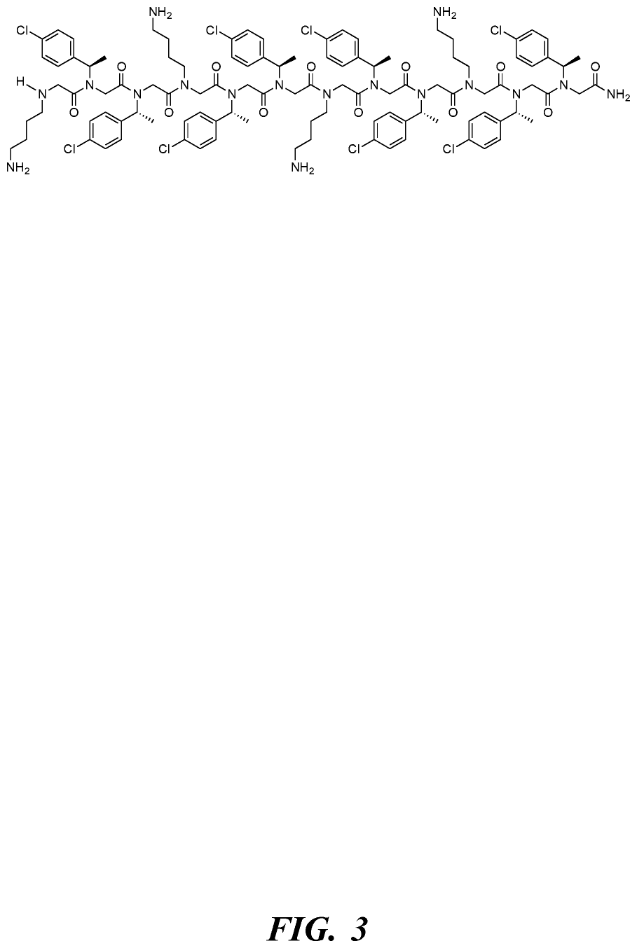

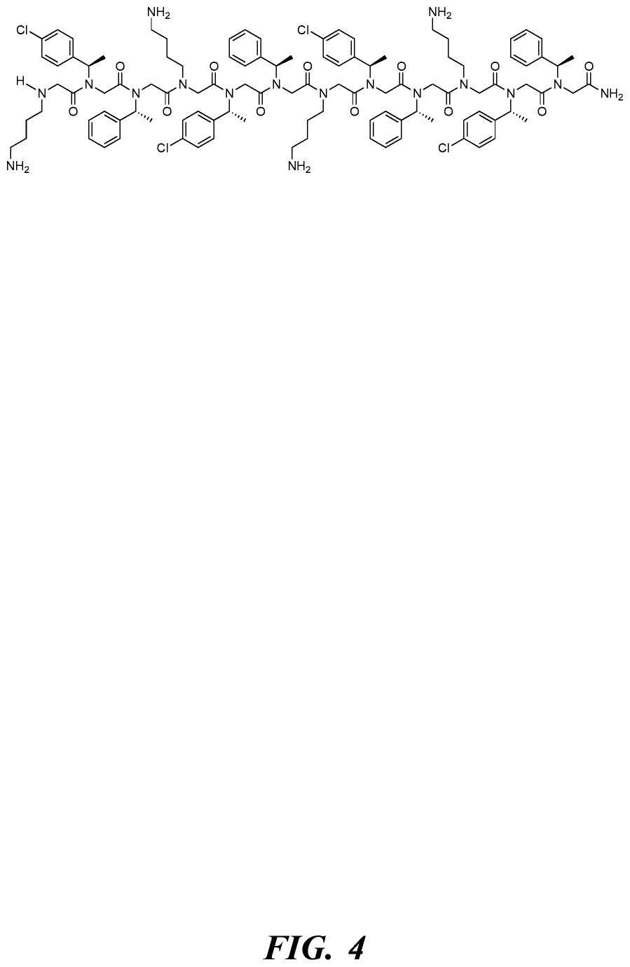

Select peptoids from EXAMPLE 1 were for activity against HSV-1 in time- and dose-response assays. These peptoids were tested at 5 and 20 μg/ml for 2 hr at 37° C. (FIG. 3) or at 20 μg/ml for 0-120′ at 37° C. (FIG. 4) prior to infection of OKF6-TERT-1 cells in triplicate. The samples were subjected to quantification as in EXAMPLE 1. The results in FIG. 1 demonstrate that the activity can be observed as early as 30′ at 20 μg/ml, and with 5 μg/ml at 2 hr incubation. Together, the results suggest that peptoids may be developed as antiviral agents against HSV-1.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and subcombinations without departing from the scope of the present disclosure. By way of example and not of limitation, the limitations of two or more dependent claims may be combined with each other without departing from the scope of the present disclosure.

What is claimed is:

1. A contraceptive device, comprising:
a surface; and
a lubricant applied to said surface, said lubricant including (a) a lubricious medium, and (b) a pharmaceutically effective amount of a peptoid disposed in said lubricious medium.

2. The contraceptive device of claim 1, wherein said lubricant includes an anionic microgel.

3. The contraceptive device of claim 2, wherein said peptoid is loaded into said microgel by complexation.

4. The contraceptive device of claim 1, wherein said peptoid is spermicidal.

5. The contraceptive device of claim 1, wherein said peptoid is antiviral.

6. The contraceptive device of claim 1, wherein said contraceptive device includes a polyacrylic-acid coated surface.

7. The contraceptive device of claim 6, wherein said peptoid is released from said polyacrylic-acid coated surface.

8. The contraceptive device of claim 6, wherein said peptoid is released from said polyacrylic-acid coated surface.

9. The contraceptive device of claim 1, wherein said peptoid comprises a poly-N-substituted glycine compound of a formula
A [symbol] X—Y—Z [symbol] ₙ—B
wherein
A is a terminal N-alkyl substituted glycine residue,
n is an integer,
B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
X, Y and Z are independently selected from the group consisting of N-substituted glycine residues, wherein said N-substituents are independently selected from the group consisting of natural α-amino acid side chain moieties, isomers and carbon homologs thereof, and proline residues.

10. The contraceptive device of claim 9, wherein said alkyl substituent is selected from about C₄ to about C₂₀ linear, branched and cyclic alkyl moieties.

11. The contraceptive device of claim 9, wherein n has a value within the range of 1-3.

12. The contraceptive device of claim 9, wherein at least one of said X, Y and Z residues is N_{Lys} and at least one said N-substituent is chiral.

13. The contraceptive device of claim 9, wherein at least one of Y and Z are proline residues.

14. The contraceptive device of claim 9, wherein Y and Z are proline residues.

15. The contraceptive device of claim 9, wherein A is a terminal N-alkyl substituted glycine residue, wherein said alkyl substituent selected from the group consisting of C₆ to about C₁₈ linear alkyl moieties, wherein B is NH₂, and wherein n is 1 or 2.

16. The contraceptive device of claim 9, wherein A is a terminal N-alkyl substituted glycine residue, said alkyl substituent selected from about C₆ to about C₁₈ linear alkyl moieties; wherein B is an N_{Lys} residue; and wherein n is 1.

17. The contraceptive device of claim 9, wherein the compound is a hexamer.

18. The contraceptive device of claim 9, wherein the compound is a dodecamer.

19. The contraceptive device of claims 9-18, and wherein at least one of A, B, X, Y and Z contains a halogen-bearing moiety.

20. The contraceptive device of claim 19, wherein said halogen-bearing moiety contains a halogen-substituted aryl moiety.

21. The contraceptive device of claim 19, wherein said halogen-bearing moiety contains a chloro-substituted aryl moiety.

22. The contraceptive device of claim 19, wherein said halogen-bearing moiety contains a bromo-substituted aryl moiety.

23. The contraceptive device of claim 19, wherein said halogen-bearing moiety contains an iodo-substituted aryl moiety.

24. The contraceptive device of claim 19, wherein each mer in the hexamer contains a halogen-substituted aryl moiety.

25. The contraceptive device of claim 19, wherein some of the mers in the hexamer contain a halogen-substituted aryl moiety, and wherein some of the mers in the hexamer contain a halogen-free aryl moiety.

26. The contraceptive device of claim 19, wherein exactly one of the mers in the hexamer contains a halogen-substituted aryl moiety.

27. The contraceptive device of claim 19, wherein each mer in the hexamer contains a halogen-substituted aryl moiety.

28. The contraceptive device of claim 19, wherein some of the mers in the hexamer contain a halogen-substituted aryl moiety, and wherein some of the mers in the hexamer contain a halogen-free aryl moiety.

29. The contraceptive device of claim 19, wherein only the first and last mers in the hexamer contain a halogen-substituted aryl moiety.

30. The contraceptive device of claim 19, wherein at least two of A, B, X, Y and Z contain a halogen-bearing moiety.

31. The contraceptive device of claim 9, wherein all of A, B, X, Y and Z contain a halogen-bearing moiety.

32. The contraceptive device of claims 9-31, wherein said pharmaceutical composition is a pharmaceutically acceptable salt of the poly-N-substituted glycine compound.

33. The contraceptive device of claim 9, wherein the poly-N-substituted glycine is H—(NLys-Nspe-Nspe)₄-NH₂.

34. The contraceptive device of claim 9, wherein the poly-N-substituted glycine is Cy5.5-Ahx-(NLys-Nspe-Nspe)₄-NH₂.

35. The contraceptive device of claim 9, wherein the poly-N-substituted glycine is H—(NLys-Nspe-Nspe(p-Br))₂—NH₂.

36. The contraceptive device of claim 9, wherein the poly-N-substituted glycine is H-Ntridec-NLys-Nspe-Nspe-NLys-NH₂.

37. The contraceptive device of claim 9, wherein the poly-N-substituted glycine is H—(NLys-Nspe-Nspe)₃-NLys-Nspe-NH₂.

38. The contraceptive device of claim 9, wherein the poly-N-substituted glycine is H—(NLys-Nspe-Nspe)₂-NH₂.

39. The contraceptive device of claim 9, wherein the poly-N-substituted glycine is H-Ndec-(NLys-Nspe-Nspe)₂-NH₂.

40. The contraceptive device of claim 9, wherein the poly-N-substituted glycine is H—Ndec-(NLys-Nspe-Nspe(p-Br))₂—NH₂.

41. The contraceptive device of claim 9, wherein the poly-N-substituted glycine is H-Ntridec-(NLys-Nspe-Nspe(p-Br))₂—NH₂.

42. The contraceptive device of claim 1, wherein said peptoid comprises a poly-N-substituted glycine compound comprising an N-terminal N-alkyl substituted glycine residue, where said alkyl substituent is selected from about C₄ to about C₂₀ linear, branched and cyclic alkyl moieties; a C-terminus selected from NH₂, one and two N-substituted glycine residues, said N-substituents independently selected from α-amino acid side chain moieties and carbon homologs thereof; and 2 to about 15 monomeric residues between said N- and C-termini, each said residue independently selected from proline residues and N-substituted glycine residues, said N-substituents independently selected from natural α-amino acid side chain moieties, isomers and carbon homologs thereof, at least one said monomeric residue is NLs and at least one said N-substituent is chiral, said monomeric residues selected to provide said compound a non-periodic sequence of monomeric residues.

43. The contraceptive device of claim 42, wherein said N-terminus is an N-alkyl substituted glycine residue, said alkyl substituent selected from about C₆ to about C₁₈ linear alkyl moieties.

44. The contraceptive device of claim 43, wherein said monomeric residues comprise 2-5 (X—Y—Z) non-periodic trimers.

45. The contraceptive device of claim 44, wherein at least one X, Y and Z in each of said trimers is selected to interrupt 3-fold periodicity.

46. The contraceptive device of claim 44, wherein said monomeric residues comprise at least two non-consecutive repeat trimers, with at least one residue therebetween.

47. The contraceptive device of claim 46, wherein at least one X in at least one said trimer is an NLys residue, and at least one of Y and Z in at least one said trimer is a proline residue.

48. The contraceptive device of claim A42, wherein at least one of said residues contains at least one halogen.

49. The contraceptive device of claims 42-48, wherein said at least one halogen is selected from the group consisting of bromine, chlorine and iodine.

50. The contraceptive device of claim 48, wherein at least one of said residues contains at least one halogen-substituted aryl moiety.

51. The contraceptive device of claim 48, wherein each of said residues contains at least one halogen-substituted aryl moiety.

52. The contraceptive device of claim 48, wherein some of said residues contain at least one halogen-substituted aryl moiety, and wherein some of said residues contain at least one non-halogenated-substituted aryl moiety.

53. The contraceptive device of claims 50-52, wherein said halogen-substituted aryl moiety is para-substituted.

54. The contraceptive device of claims 50-52, wherein said halogen-substituted aryl moiety is perhalogenated.

55. The contraceptive device of claims 42-54, wherein the pharmaceutical composition is a pharmaceutically acceptable salt of the poly-N-substituted glycine compound.

56. The contraceptive device of claims 1-55, wherein the contraceptive device is a condom including a sheath, and wherein said pharmaceutical composition is applied to said sheath.

57. The contraceptive device of claim 1, wherein said surface is an elastomeric surface.

58. A method for preventing the transmission of sexually transmitted diseases during sexual intercourse between two sexual partners, comprising:
providing a lubricant including (a) a lubricious medium, and (b) a pharmaceutically effective amount of a peptoid disposed in said lubricious medium; and
applying the lubricant to (a) a contraceptive device, or (b) the genitalia of at least one of the partners.

59. The method of claim 58, wherein the peptoid comprises a poly-N-alkyl substituted glycine compound of a formula
H—N_{R} [symbol] X—Y—Z [symbol] ₙ—B
wherein
B is selected from NH₂ and X′,
N_{R}, X, Y, Z and X′ are independently selected from N-substituted glycine residues containing N-substituents, wherein said N-substituents of said N-substituted glycine residues are independently selected from natural α-amino acid side chain moieties, isomers and carbon homologs thereof, and proline residues,
n is an integer, and
R is an N-alkyl substituent of said N_{R} glycine residue, said substituent selected from about C₄ to about C₂₀ linear, branched and cyclic alkyl moieties.

60. The method of claim 59, wherein n is 2 and B is NH₂.

61. The method of claim 59, wherein n is 1 and B is X′.

62. The method of claim 61, wherein at least one of X and X′ are N_{Lys} residues.

63. The method of claim 62, wherein said N-alkyl substituent is selected from about C₆ to about C₁₈ linear, branched and cyclic alkyl moieties.

64. The method of claim 63, wherein X and X′ are N_{Lys} residues.

65. The method of claim 63 of a formula
H—N_{tridec}—N_{Lys}—Nₛₚₑ—Nₛₚₑ—N_{Lys}—NH₂.

66. The method of claim 59, wherein said alkyl substituent is selected from about C₄ to about C₂₀ linear, branched and cyclic alkyl moieties.

67. The method of claim 59, wherein n has a value within the range of 1-2.

68. The method of claim 59, wherein at least one of said X, Y and Z residues is N_{Lys} and at least one said N-substituent is chiral.

69. The method of claim 59, wherein at least one of Y and Z are proline residues.

70. The method of claim 59, wherein Y and Z are proline residues.

71. The method of claim 59, wherein A is a terminal N-alkyl substituted glycine residue, wherein said alkyl substituent selected from the group consisting of C₆ to about C₁₈ linear alkyl moieties, wherein B is NH₂, and wherein n is 1 or 2.

72. The method of claim 59, wherein N_{R} is a terminal N-alkyl substituted glycine residue, said alkyl substituent selected from about C₆ to about C₁₈ linear alkyl moieties; wherein B is an N_{Lys} residue; and wherein n is 1.

73. The method of claim 59, wherein the compound is a hexamer.

74. The method of claim 59, wherein the compound is a dodecamer.

75. The method of claims 59-64, wherein at least one said N-substituent contains a halogen atom.

76. The method of claim 64, wherein said halogen-bearing moiety contains a halogen-substituted aryl moiety.

77. The method of claim 64, wherein said halogen-bearing moiety contains a chloro-substituted aryl moiety.

78. The method of claim 64, wherein said halogen-bearing moiety contains a bromo-substituted aryl moiety.

79. The method of claim 64, wherein said halogen-bearing moiety contains an iodo-substituted aryl moiety.

80. The method of claim 64, wherein each mer in the hexamer contains a halogen-substituted aryl moiety.

81. The method of claim 64, wherein some of the mers in the hexamer contain a halogen-substituted aryl moiety, and wherein some of the mers in the hexamer contain a halogen-free aryl moiety.

82. The method of claim 64, wherein exactly one of the mers in the hexamer contains a halogen-substituted aryl moiety.

83. The method of claim 64, wherein each mer in the hexamer contains a halogen-substituted aryl moiety.

84. The method of claim 64, wherein some of the mers in the hexamer contain a halogen-substituted aryl moiety, and wherein some of the mers in the hexamer contain a halogen-free aryl moiety.

85. The method of claim 64, wherein only the first and last mers in the hexamer contain a halogen-substituted aryl moiety.

86. The method of claim 64, wherein at least two of N_{R}, X, Y, Z and X′ contain a halogen-bearing moiety.

87. The method of claim 64, wherein all of N_{R}, X, Y, Z and X′ contain a halogen-bearing moiety.

88. The method of claim 59, wherein the pharmaceutical composition is a pharmaceutically acceptable salt of the poly-N-substituted glycine compound of claims B2-B30.

89. The method of claim 59-88, wherein the contraceptive device is a condom including a sheath, and wherein said pharmaceutical composition is applied to said sheath.
